(19) United States
(12) Patent Application Publication
(10) Pub. No.: US 2006/0225344 A1
(43) Pub. Date: Oct. 12, 2006

(54) Identification labels in plants or plant parts

(76) Inventors: Wilhelmus Maria Van Der Krieken, Wageningen (NL); Cornelis Johannes Kok, Nijmegen (NL)

Correspondence Address: Christine A Lekutis;Medlen & Carroll, Suite 350, 101 Howard Street, San Francisco, CA, 94105, US

(21) Appl. No.: 10/543,132

(22) PCT Filed: Jan. 24, 2003
(86) PCT No.: PCT/EP03/00785

§ 371 Date: Apr. 17, 2006

(51) Int. Cl. G01V 15/00; A01G 7/00; A01H 3/00

(52) U.S. Cl. 47/58.1 R; 504/114; 504/116.1; 800/295

(57) ABSTRACT

The invention relates to a method for providing plants and/or plant parts with an identification label, comprising contacting the plant or plant part with a product, comprising one or more types of tracer molecules, preferably fluorescent tracer molecules, and allowing the plant or plant part to take up the tracer molecules either inside the plant or plant parts or on the surface thereof. The invention further relates to products for use in the method and plant and plant parts treated with the method and to methods for identifying plants or plant parts carrying an identification label.

The present invention relates to a method for providing plants and/or plant parts with an identification label, to plants or plant parts carrying an identification label, to methods for identifying such plants or plant parts and to products for use in the provision of an identification label.

In many cases it is desirable to identify batches of plants according to their origin, their time of production, or to know the owner of plant material. Tracking and tracing of plant material throughout the production system would provide the possibility of a better quality control. In addition, it may be important to identify batches of plant material that have received special treatments.

At present, batches of plants can be provided with a paper label showing information regarding country of origin, grower, date of harvest etc. However, paper labels can be lost or changed and, because they are not physically attached to a particular plant, it is never certain that the information regarding the batch to which the label is attached is indeed accurate.

It is therefore an object of the present invention to provide an alternative method that allows for the identification of plants and plant parts.

This is achieved according to the invention by a method for providing plants and/or plant parts with an identification label, comprising contacting the plant or plant part with a product comprising one or more types of tracer molecules, preferably fluorescent tracer molecules, and allowing the plant or plant part to take up the tracer molecules either inside the plant or plant part or on the surface thereof. The product can be a liquid or a solid, in particular a powder.

The identification label thus consists of one or more tracer molecules that each emit a different colour that corresponds with a particular property of the plant or plant part. By visualizing the label the plant can be identified as having the property corresponding to that colour. By using more than one colour a plant can be provided with an identification label that corresponds with a set of properties.

The tracers of choice are fluorescent tracers that are easy to detect and stable over a prolonged period of time. Fluorescent tracers can for example be chosen from a large group of fluorescent compounds, that are preferably non-toxic and environmentally sound, and among which optical brighteners and quantum dots are preferred.

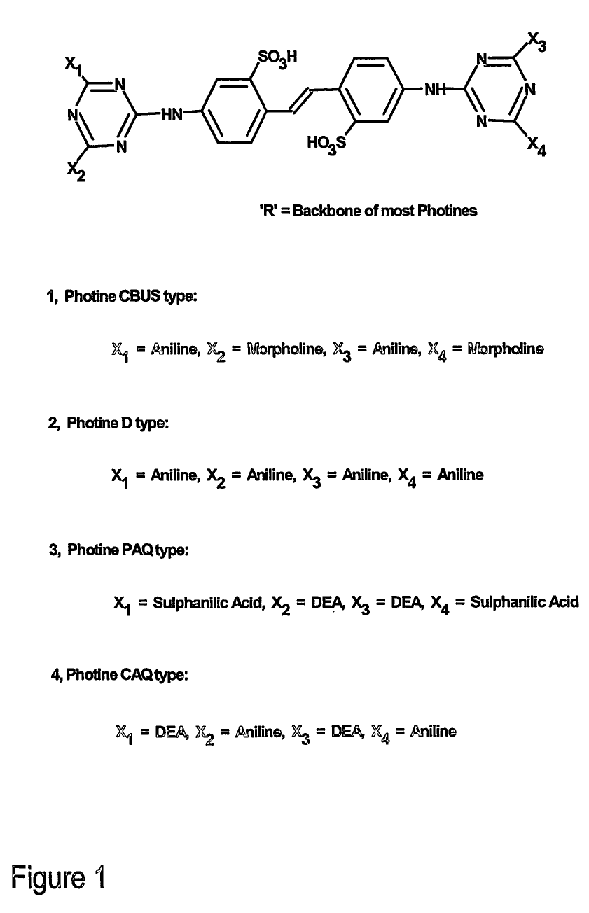

Optical brighteners reflect ultraviolet light as white-blue light. Optical brighteners have the advantage that they are relatively stable to thermal and biological degradation, safe, well tested and allowed in many different applications, such as washing powders, tooth paste, wood, paper etc. Furthermore these compound are easy to detect with simple means, both in solution and after uptake in the plant material or application on the surface thereof. They cannot be seen by the unaided eye. The person skilled in the art is very well capable of selecting suitable optical brighteners for use in the invention. Examples are Photine® CBUS, Photine® D, Photine® PAQ and Photine® CAQ (FIG. 1).

Quantum dots are nanometre (10⁻⁹ metre) scale particles that are neither small molecules nor bulk solids. Their composition and small size (a few hundred to a few thousand atoms) give these dots extraordinary optical properties that can be readily customized by changing the size or composition of the dots. Quantum dots absorb light, then quickly re-emit the light but in a different colour. Quantum dots are fluorophores that are bright, non-photobleaching and have narrow, symmetric emission spectra. They come in multiple resolvable colours that can be visualised by exciting them simultaneously using a single excitation wavelength. The colour of quantum dots—both in absorption and emission—can be “tuned” to any chosen wavelength by simply changing their size to obtain colours that span the spectrum, from ultraviolet to infrared. Quantum dots have the advantage that they have very intense fluorescence (enabling highly sensitive detection) and can be produced in millions of different colours, which allows specific labelling and detection of plant material. Quantum dots, also known as semiconductor nanocrystal compounds, are described in U.S. Pat. No. 5,990,479 to Weiss et al., issued Nov. 23, 1999 and are commercially available from the Quantum Dot Corporation (QDC) as Q-dots@.

Other fluorescent compounds that can be used in the invention are for instance 1,5-naphthalene disulfonic acid disodium salt, 2-amino-1-naphthalene sulfonic acid, 5-amino-2-naphthalene sulfonic acid, 4-amino-3-hydroxyl-1-naphthalene sulfonic acid, 6-amino-4-hydroxyl-2-naphthalene sulfonic acid, 7-amino-1,3-naphthalene disulfonic acid, potassium salt, 4-amino-5-hydroxy-2,7-naphthalene disulfonic acid, 5-dimethylamino-1-naphthalene sulfonic acid, 2,6-naphthalene dicarboxylic acid, dipotassium salt, 2-anthracene sulfonic acid, sodium salt, quinoline, 1-ethylquinaldinium iodide, dibenzofuran sulfonic acid, cresyl violet acetate, bathophenanthroline disulfonic acid disodium salt, 1-amino-4-naphthalene sulfonic acid, 1-amino-7-naphthalene sulfonic acid, amino 2,5-benzene disulfonic-acid, 1,3,6,8-pyrenetetra sulfonic acid, tetrasodium salt, 8-hydroxy-1,3,6-pyrene trisulfonic acid, trisodium salt, 3,4,9,10-perylene tetracarboxylic acid, bis-N-methylacridinium, 2-(4-aminophenyl)-6-methylbenzothiazole, resazurin, fluorescein; or fluorescent tracers with CAS registration numbers 2391-30-2, 477-73-6, 1562-90-9, 1829-00-1, 56509-06-9, 16470-24-9, 2694-95-4, 169762-28-1, 144470-48-4, 12270-53-0, 12270-53-0, 1968-72-7, 68444-86-0, 205265-33-4, 37299-86-8, 2321-07-5, 50-82-3, 2538-84-3, 65-61-2, 52237-03-3, 27344-41-8, 6416-68-8 and their ammonium, potassium and sodium salts.

These tracers can be taken up into the plant or plant arts or can remain on the outside, as required. Detection of the tracer inside or on the surface of the plant or plant part can be performed by direct viewing upon illuminating the plant material with a light of adequate wavelength, or, more sensitively, by laser irradiation and detection of fluorescence by CCD-camera. This procedure can be made (semi) quantitative by calibration tools, based on known amounts of fluorescent tracers in the specific plant material.

The fluorescent tracers can be used in the form of powders, liquids, dispersions, slurries or solutions, as required by the application. Typically application methods for fluorescent tracers are spraying on plants or plant parts, or mixing with (pre)treatment media with or without other active compounds, for uptake by or absorption to the plant material to be labelled (seeds, fibres, stems, leaves, flowers, roots, tubers, cuttings of plants and all other means of vegetative plant propagation) for example in the vase water. Other ways of contacting are immersing, dipping dusting and coating.

The amount of fluorescent tracer to be used is typically very low. For example, the amount of Photine® PAQ solution lies between 10 and 500 μl/L.

The method of the invention can be used for a wide range of applications in order to provide whole plants or plant parts, such as cut flowers, seeds, fruits etc. with an identification label that can comprise more or less extensive information about the product carrying the label.

The identification label can for instance give information on the origin of the plant or plant part by means of differently coloured fluorescent tracers for the country of origin, the place of origin and the grower. In addition, another colour can be used to indicate the date or period of harvest of the fruit or cutting of the flower. This way the origin and production date of the product can be traced back by simple illumination of the product, which is important when a guaranteed vase life or shelf life is given for the product.

Many cut flowers come with an amount of nutrients to be added to the vase water. By adding a fluorescent tracer to the flower food the retailer can check whether a complaining customer has used the flower food provided with the flowers by means of simple illumination of the flower.

Plant or plant parts and in particular cut flowers can undergo various treatments to extend their vase life or shelf life. Addition of fluorescent tracers according to the invention to the treatment product can provide the guarantee that the treatment did actually take place. The identification label shows to which treatment(s) the plant or plant part was subjected. Such treatments include for example the prevention of leaf yellowing, prevention of vascular plugging of cut flower stems by bacteria, prevention of ethylene damage, reduction of stem growth, like for instance in tulips. In other applications such treatments comprise for example induction of root formation, such as in plant cuttings, induction of flower formation and grafting.

Another application of the method of the invention is to mark the use of certain pesticides, such as herbicides, nematicides, fungicides, insecticides, acaricides, molluscicides, preferably pesticides of natural origin. After mixing these pesticides with fluorescent tracers, application thereof can be detected in or on the plant material. It is furthermore possible to add an additional tracer corresponding to the date or period of treatment. Such identification label is for example important for quality control of the agricultural production process, especially for certification of certain defined production methods of organic farming (eco-labelling).

Yet another use of the method of the invention is to detect early leaf infection by plant pathogenic fungi. Fungi what can be visualized by means of the invention are for example leaf pathogenic fungi like Botrytis and Phytophthora, rust fungi, e.g. Puccinia, smut fungi, e.g. Ustilago, mildew, e.g. Erisyphe, false mildew, e.g. Mycosphaerella. After application to the plant (typically by spraying) the fluorescent tracers accumulate at infection sites. These infections sites can then be visualized. This technique can be used for prevention of spread of disease (by removing infected plants) or to optimize pesticide application, resulting in reduced pesticide use and/or reduced plant damage. This technique is especially advantageous when the fluorescent tracer is formulated together with natural crop protection products (NCP's) that are used as a preventive measure. In this way the use of the NCP can be detected, as well as occurrence of fungal infection despite application of the product. Fungal infection can be easily distinguished from the background level of fluorescent tracer(s), because of the accumulation of the fluorescent tracer(s) on the infection site.

Another use of the method of the invention is to indicate the status of genetic modification. Presently, batches of plant derived products, such as corn, soya etc., may consist of mixtures of genetically modified and not genetically modified products. This is undesirable in cases where the use of the genetically modified products is not wanted. When genetically modified plants or plant parts carry an identification label according to the invention, contamination of a product with genetically modified material can be traced by simple illumination.

Furthermore, the invention is useful in the protection of plant breeder's rights, because the identification label of the invention will still be visible in plants grown from plant cuttings from plants that carry an identification label according to the invention thus providing proof of illegal propagation.

A typical advantage of the method of the invention is that the plant material can be treated for a limited time, while the fluorescent tracer can be detected in the plant material throughout the production chain (from grower to consumer). It was found that the tracer remains in or on the plant material for a prolonged period of time.

The fluorescent tracers can be taken up in the plants by means of water transport within the plant or plant part. This is for example the case in cut flowers. However, uptake is not always necessary because in cut flowers the tracer will also be visible on the stem that was in contact with the vase water. When treatments solutions are sprayed on leaves or fruits or flowers the tracers are not always taken up but remain on the surface and can be visualized there. The same applies to seeds. The tracers and optionally the treatment solution will only partially pass the seed coat and remain on the outside. For the superficial application of tracers optical brighteners are very suitable because they adhere very well to fibres.

The technique described can be applied to both fresh and dried plant material. Dried plant material like seeds, fibres, stems, leaves and flowers sometimes has to be rehydrated before detection of fluorescent tracers, such as in the case of Photine® type tracers.

The invention further relates to a method for identifying a plant or plant part carrying an identification label, consisting of one or more types of fluorescent tracer molecules that are present in or on the plant or plant part and can be provided by means of the method of the invention, which identification method comprises visualization of the fluorescent tracer(s).

Visualisation and detection of the fluorescent tracer(s) in or on the plant material or in the (pre)treatment solution is simplest performed by use of a hand-held device, such as a black-light. The plant material or (pre)treatment solution lights up under the black-light in specific colours, depending on the fluorescent tracer applied. The detection of a specific tracer can be performed by the use of filters that transmit only the wavelength of maximal emission of the specific tracer. Detection of fluorescent tracers can be more sensitively performed by using laser irradiation of the plant material at the specific excitation frequency of the fluorescent tracer applied. The emitted light can be very sensitively detected by a camera device (for instance a CCD-camera). Detection and quantification of the fluorescent tracer in the (pre)treatment solution can be performed sensitively by fluorimetry. This latter option is important for process and quality control.

The invention also relates to the plant and plant parts provided with an identification label according to the invention.

According to a further aspect thereof, the invention relates to a product for providing a plant or plant part with an identification label, which product comprises one or more types of tracer molecules, preferably fluorescent tracer molecules. Preferably the product contains one or more optical brighteners and/or quantum dots as fluorescent tracer.

The product can also be a treatment product, in particular a liquid or a solid, that further comprises compounds for use in the prevention of leaf yellowing, vascular plugging of cut flower stems by bacteria, ethylene damage, in the reduction of stem growth, in the induction of root formation, such as in plant cuttings, in the induction of flower formation or in grafting or a product providing nutrients to plants and plant parts, such as flower food for cut flower.

The product can be a liquid or a solid. The liquid can for example be a solution, a dispersion, slurry etc. The solid can for instance be a powder that is used as such or is first dissolved or dispersed in water or another solvent.

In a particular embodiment, the invention relates to flower food labelled with one or more optical brighteners and/or quantum dots. Flower food may be in the form of a powder or liquid that is added to the vase water.

“Identification label” as used in this application is intended to encompass every set of fluorescent tracers that is present on or in a plant or plant part and can be used upon visualization to detect certain properties of the plant or plant part. A “set” means one or more tracers. “Properties” in this sense does not necessarily mean genotypic or phenotypic properties, although in certain embodiments, such as for checking whether the plant or plant part is the product of genetic modification, such properties may be intended. “Properties” may also mean information about the plant or plant part. Such information may refer to something that is physically linked to the plant or plant part, i.e. present in or on the plant or plant part, such as treatment compounds or solutions, or infective organisms etc., or not physically linked, such as country of origin, grower, date of harvest etc.

“Tracer molecules” and “tracers” are used interchangeably and relate to all compounds described in this application and any other compound not explicitly mentioned but still capable of fulfilling the role of a tracer as described herein.

“Plants” are complete plants, including the roots, leaves and stem, and including shrubs, trees etc. “Plant parts” are all materials originating from a plant, such as seeds, fruits, flowers, stems, cuttings, leaves, grains, heads, roots, etc., either intact or processed (cut, grinded, sliced, mixed with other compounds etc.). Plants and plant parts may also be named “plant material”.

The invention will be further illustrated in the examples that follow and that are not intended to limit the invention in any way.

In the Example reference is made to the following figures:

FIG. 1: Structural formula's of different Photine® type fluorescent tracers.

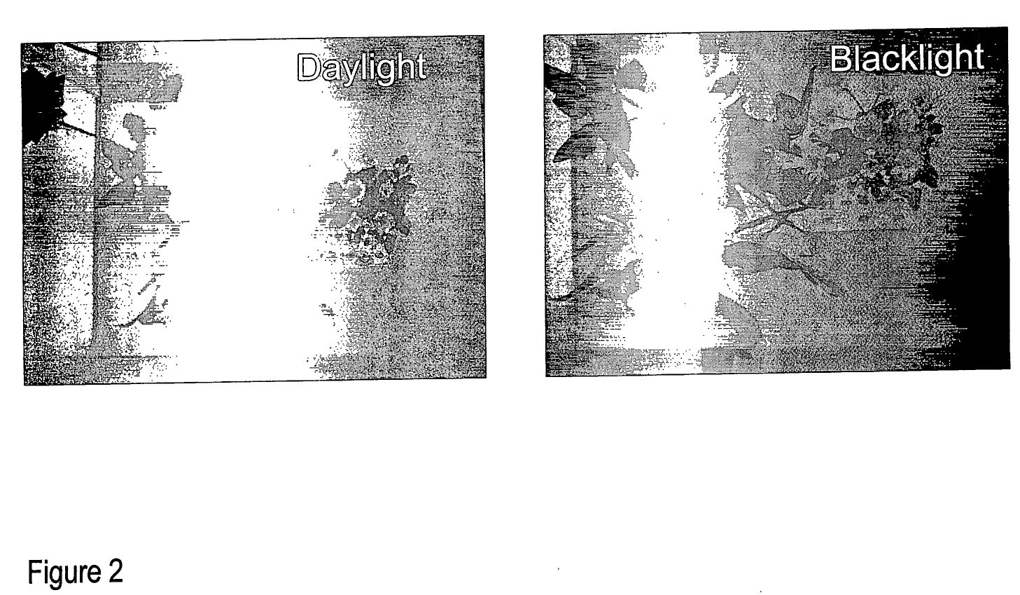

FIG. 2: Overview of the experiment of Example 1. The flowers were photographed 2 days after the treatment. The left panel shows illumination with daylight only. The right panel shows the flowers upon daylight and black-light illumination.

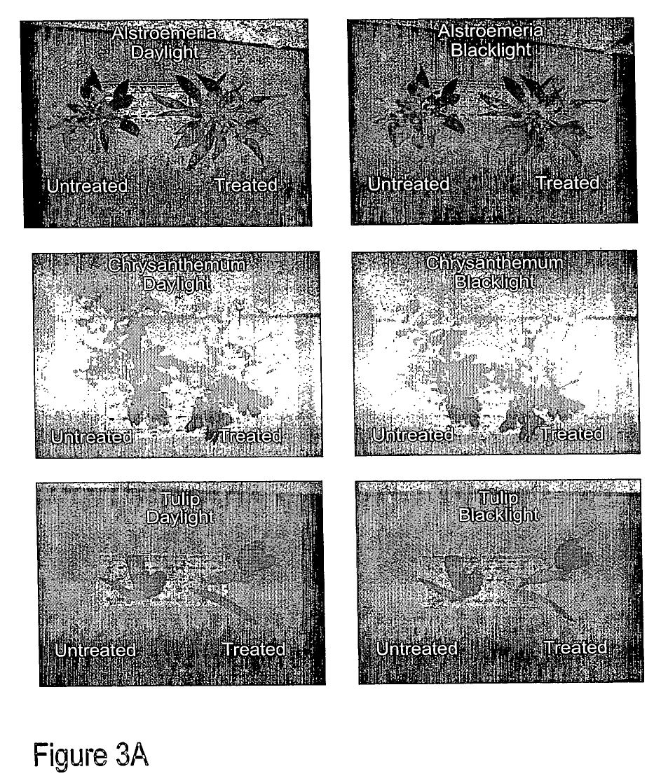

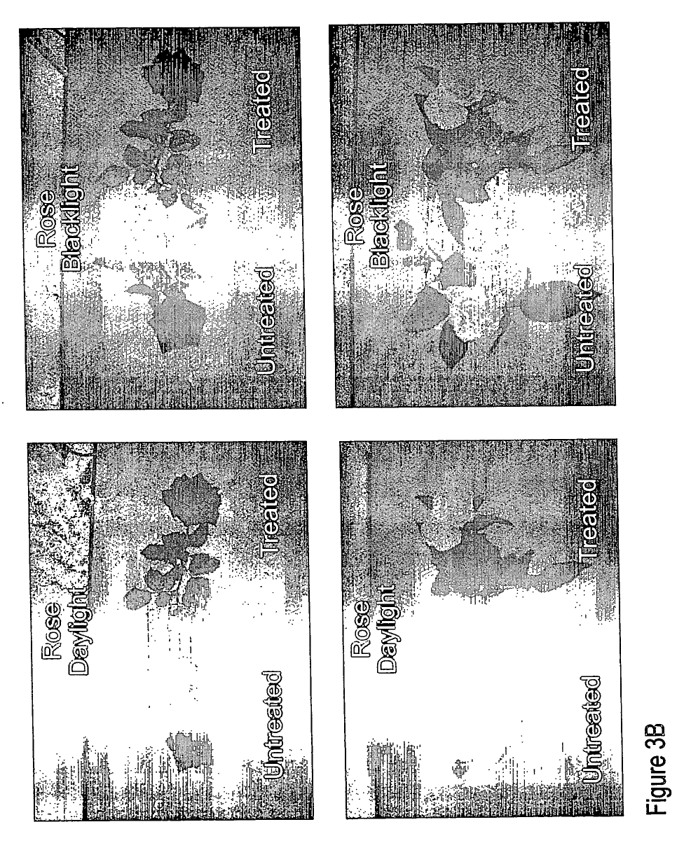

FIG. 3A: Alstroemeria (top panels), Chrysanthemum (middle panels) and Tulip (bottom panels) treated with the optical brightener Photine® PAQ. Left panels are illuminated with daylight, right panels with both daylight and black-light. Fluorescence can be seen in the right panels as lighter (blue) areas indicated with an arrow.

FIG. 3B: Red (top panels) and white (bottom panels) rose flowers treated with the optical brightener Photine® PAQ. Left panels are illuminated with daylight, right panels with both daylight and black-light. Fluorescence can be seen in the right panels as lighter (blue) areas indicated with an arrow.

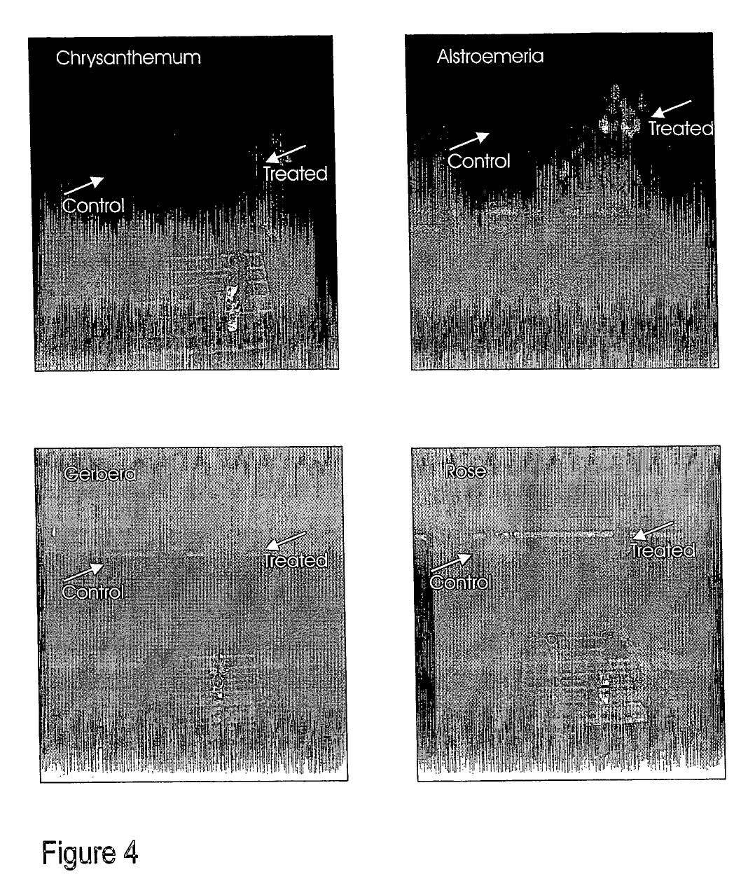

FIG. 4: Chrysanthemum, Alstroemeria, Gerbera and Rose treated with Photine® PAQ and illuminated by black-light. Control plants can hardly be seen and show no fluorescence, whereas treated plants are visible.

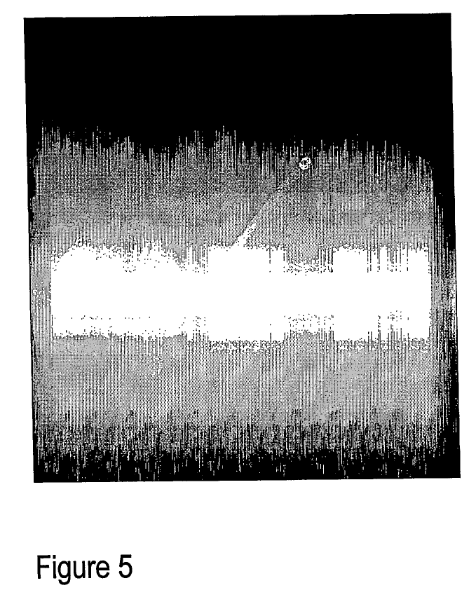

FIG. 5: Flower stem treated with Photine® PAQ and illuminated with black-light. Both the cut and outside of the stem show fluorescence.

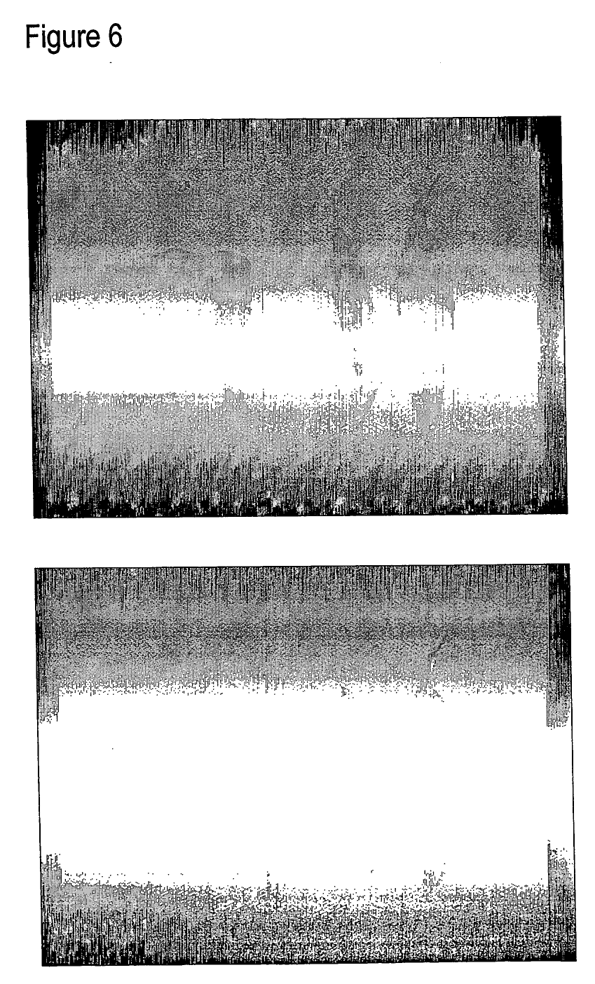

FIG. 6: Fluorescence on rehydrated dried leaves of Alstroemeria treated with Photine® PAQ.

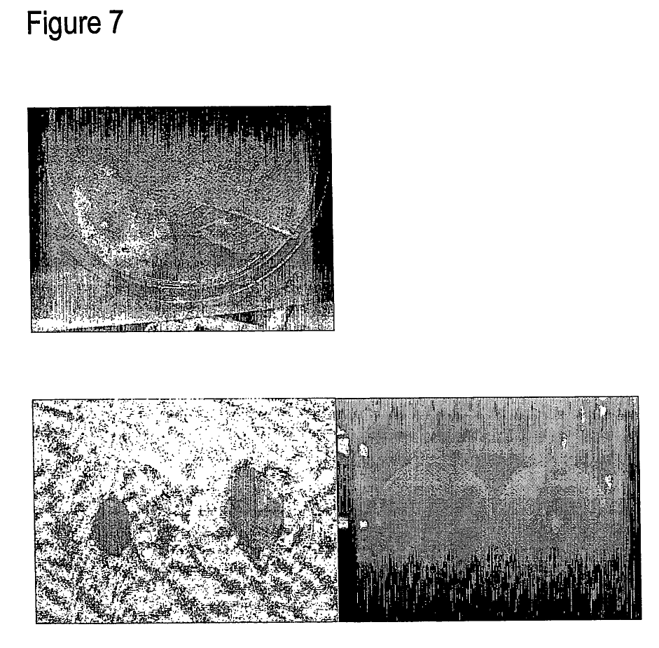

FIG. 7: Fluorescence on infected and uninfected leaves of potato after treatment with Photine® CAQ.

### EXAMPLES

### Example 1

Fluorescent Tracers in Cut Flowers

1. Introduction

An important field of application of the present invention is monitoring the flower production chain (from grower via packer and retailer to consumer). Cut flowers can be (pre)treated with a number of chemical compounds to prevent leaf yellowing or vascular plugging of cut flower stems by bacteria, to provide nutrients to the flowers, to extend vase life, to prevent ethylene damage or to reduce stem growth. According to the invention, the fluorescent tracer (FT) is applied to the medium along with the other pre-treatment compounds. It is transported into the plant stem and plant leaves actively or passively along with the water transport. This experiment demonstrates that fluorescence is visible in the plant leaves and flowers.

2. Materials and Methods

Four types of flowers (Gerbera, Alstroemeria, Rose and Chrysanthemum) were treated with water containing 100 μl/l of Photine® PAQ. After 2 days a picture was taken, showing the fluorescence in the leaves and flowers on irradiation with black-light (FIGS. 2-4). Without black-light illumination, there is no visible difference between the treated flowers and the control treatment (only water). With black-light illumination, lighter (blue) areas are visible on the leaves and flowers. Fluorescence could also be detected on the outside of the stem and on the cut (FIG. 5). No toxicity to the plants could be detected.

After one month fluorescence was still visible in fresh leaves.

Similar results were found for Q-dots® (results not shown). Different colours of Q-dots® gave comparable efficacy.

### Example 2

Fluorescence in Dried and Rehydrated Leaves

This experiment was performed with Alstroemeria leaves. The cut flowers were treated with Photine® PAQ as described for example 1. After 6 weeks, dried leaves were subjected to black-light illumination. No fluorescence was observed in this case. However, the presence of Photine® PAQ could be easily detected by fluorescence under black-light after rehydration for 5 minutes. FIG. 6 shows leaves of Alstroemeria illuminated by daylight (left-hand panel) en black-light (right-hand panel). The dried leaf is shown on the left, the two rehydrated leaves are shown on the right. Leaves were taken from cut flowers treated as described in example 1. Light areas (blue fluorescence) in the leaves shows presence of Photine® PAQ.

### Example 3

Detection of Botrytis and Phytophthora with Photine® CAQ

The fungus Botrytis cinerea was grown on a Petri dish containing nutrient agar with 40 μl/L Photine® CAQ (see FIG. 1). The mycelium of the fungus showed very strong fluorescence (results not shown), indicating accumulation of the fluorescent tracer in the mycelium or on the fungal cell walls.

Infections of the fungus Phytophthora on potato leaves treated with Photine® CAQ solution (100 μl/l water) showed very strong fluorescence, while the uninfected leaf area showed only a weak background fluorescence. FIG. 7 shows Phytophthora-infected potato leaves illuminated with daylight (left-hand picture of bottom row) or black-light (right-hand picture of bottom row). In each picture the leaf in the right-hand Petri dish was treated with Photine® CAQ. Blue fluorescence (lighter areas) in the Photine® PAQ treated leaf shows infection with Phytophthora. The upper picture in FIG. 7 shows that in Phytophthora-infected potato leaves illuminated with black-light, no fluorescence is visible in absence of fluorescent tracer.

### Example 4

Detection of Propagation of Rose and Tomato, Labelled with Q-Dots® or Photine® CAQ

Cut stems of rose and tomato were incubated with solutions containing 0.01% Photine® CAQ or 0.001% Q-dots®. After 24 hours scions were taken from these cuttings, which were subsequently treated with root powder and put in rock wool, according to the standard procedure for vegetative propagation of tomato and rose. After 4 weeks, fluorescence by Photine® CAQ was detected by black-light illumination and fluorescence of the Q-dots® was detected by CCD camera after excitation with laser light of the appropriate wavelengths (as described on the website http://www.qdots.com). It was found that the treatment of the parent material with fluorescent tracers could still be detected in the propagated plants.

### Example 5

Labelling of seeds with Photine® CAQ or Q-dots®

Dried seeds were treated with solutions containing 0.01% Photine® CAQ or 0.001% Q-dots®, for 3 hours. After this the seeds were re-dried at room temperature. Fluorescence of seeds was detected after rehydration for 1 hour. Fluorescence by Photine® CAQ, as detected by black-light illumination or fluorescence of the Q-dots®, detected by CCD camera after excitation with laser light (see http://www.qdots.com) did not decrease for a period of 2 months. Different colours of Q-dots® were equally effective.

1. Method for providing plants and/or plant parts with an identification label, comprising contacting the plant or plant part with a product, comprising one or more types of tracer molecules, preferably fluorescent tracer molecules, and allowing the plant or plant part to take up the tracer molecules either inside the plant or plant parts or on the surface thereof.

2. Method as claimed in claim 1, wherein the product is a liquid.

3. Method as claimed in claim 2, wherein contacting the plant or plant part with the product is performed by spraying, immersing, dipping or coating the plant or plant parts with the liquid or putting the plant or plant parts in a container holding the liquid.

4. Method as claimed in claim 1, wherein the product is a powder.

5. Method as claimed in claim 4, wherein contacting the plant or plant part with the product is performed by dusting with the powder or dissolving the powder in water to obtain a liquid that can be used for spraying, immersing, dipping, or coating the plant or plant part or can be put in a container holding the plant or plant parts.

6. Method as claimed in claim 3, wherein the container holding the liquid and the plant or plant parts is a vase with water comprising the tracer molecules.

7. Method as claimed in claim 1, wherein the identification label is intended to show the origin of the plant or plant part.

8. Method as claimed in claim 1, wherein the identification label is intended to show the date on which a plant or plant part was cut or harvested.

9. Method as claimed in claim 1, wherein the identification label is intended to show that the plant or plant part was subjected to a treatment.

10. Method as claimed in claim 9, wherein the treatment is selected from the group consisting of pesticide treatment, treatment for preventing leaf yellowing, treatment to prevent vascular plugging of cut flowers, treatment to prevent ethylene damage, treatment to reduce stem growth, treatment to induce root formation, treatment to induce flower formation, treatment to extend the vase life of cut flowers and grafting.

11. Method as claimed in claim 1, wherein the identification label is intended to show that nutrients were provided to cut flowers.

12. Method as claimed in claim 1, wherein the identification label is intended to show the presence on the plant or plant part of an infection site.

13. Method as claimed in claim 12, wherein the infection is caused by an organism selected from the group consisting of leaf pathogenic fungi, rust fungi, smut fungi, mildew, and false mildew.

14. Method as claimed in claim 1, wherein the identification label is intended to show that the plant or plant part was genetically modified.

15. Method as claimed in claim 1, wherein the identification label is intended to show that the plant was vegetatively propagated from a parent plant carrying the label.

16. Method for identifying a plant or plant part carrying an identification label, consisting of one or more types of fluorescent tracer molecules, comprising visualization of the label with a source of light.

17. Method as claimed in claim 16, wherein the source of light is selected from black-light, and laser.

18. Method as claimed in claim 16, further comprising registration of the light emitted by the one or more fluorescent tracers.

19. Method as claimed in claim 1, wherein the tracer molecule is a compound selected from the group consisting of PHOTINE CBUS, PHOTINE D, PHOTINE PAQ, PHOTINE CAQ, 1,5-naphthalene disulfonic acid disodium salt, 2-amino-1-naphthalene sulfonic acid, 5-amino-2-naphthalene sulfonic acid, 4-amino-3-hydroxyl-1-naphthalene sulfonic acid, 6-amino-4-hydroxyl-2-naphthalene sulfonic acid, 7-amino-1,3-naphthalene disulfonic acid, potassium salt, 4-amino-5-hydroxy-2,7-naphthalene disulfonic acid, 5-dimethylamino-1-naphthalene sulfonic acid, 2,6-naphthalene dicarboxylic acid, dipotassium salt, 2-anthracene sulfonic acid, sodium salt, quinoline, 1-ethylquinaldinium iodide, dibenzofuran sulfonic acid, cresyl violet acetate, bathophenanthroline disulfonic acid disodium salt, 1-amino-4-naphthalene sulfonic acid, 1-amino-7-naphthalene sulfonic acid, amino 2,5-benzene disulfonic-acid, 1,3,6,8-pyrenetetra sulfonic acid, tetrasodium salt, 8-hydroxy-1,3,6-pyrene trisulfonic acid, trisodium salt, 3,4,9,10-perylene tetracarboxylic acid, bis-N-methylacridinium, 2-(4-aminophenyl)-6-methylbenzothiazole, resazurin, and fluorescein.

20. Plant or plant part carrying an identification label, consisting of one or more types of fluorescent tracer molecules.

21. Plant or plant part as claimed in claim 20, wherein the fluorescent tracer molecule is a compound selected from the group consisting of PHOTINE CBUS, PHOTINE D, PHOTINE PAQ, PHOTINE CAQ, 1,5-naphthalene disulfonic acid disodium salt, 2-amino-1-naphthalene sulfonic acid, 5-amino-2-naphthalene sulfonic acid, 4-amino-3-hydroxyl-1-nalphthalene sulfonic acid, 6-amino-4-hydroxyl-2-nalphthalene sulfonic acid, 7-amino-1,3-naphthalene disulfonic acid, potassium salt, 4-amino-5-hydroxy-2,7-naphthalene disulfonic acid, 5-dimethylamino-1-naphthalene sulfuric acid, 2,6-naphthalene dicarboxylic acid, dipotassium salt, 2-anthracene sulfonic acid, sodium salt, quinoline, 1-ethylquinaldinium iodide, dibenzofuran sulfonic acid, cresyl violet acetate, bathophenanthroline disulfonic acid disodium salt, 1-amino-4-naphthalene sulfonic acid, 1-amino-7-naphthalene sulfonic acid, amino 2,5-benzene disulfonic-acid, 1,3,6,8-pyrenetetra sulfonic acid, tetrasodium salt, 8-hydroxy-1,3,6-pyrene trisulfonic acid, trisodium salt, 3,4,9,10-perylene tetracarboxylic acid, bis-N-methylacridinium, 2-(4-aminophenyl)-6-methylbenzothiazole, resazurin, and fluorescein.

22. Plant or plant part produced by a method comprising, contacting a plant or plant part with a product, comprising one or more types of tracer molecules, and allowing the plant or plant part to take up the tracer molecules either inside the plant or plant parts or on the surface thereof.

23. Product for providing a plant or plant part with an identification label, which product comprises one or more types of tracer molecules.

24. Product as claimed in claim 23, wherein the tracer molecules are selected from optical brighteners and quantum dots.

25. Product as claimed in claim 23, further comprising one or more treatment compounds for pesticide treatment, treatment for preventing leaf yellowing, treatment to prevent vascular plugging of cut flowers, treatment to prevent ethylene damage, treatment to reduce stem growth, treatment to induce root formation, treatment to induce flower formation, treatment to extend the vase life of cut flowers, or grafting.

26. Product as claimed in claim 25, which product is flower food.

27. Product as claimed in claim 23, for use in showing the origin of the plant or plant part, showing the date on which a plant or plant part was cut or harvested, showing that the plant or plant part was subjected to a treatment, showing that nutrients were provided to cut flowers, or showing the presence on the plant or plant part of an infection site.

28. Method as claimed in claim 1, wherein said tracer molecules are fluorescent tracer molecules.

29. Method as claimed in claim 5, wherein the container holding the liquid and the plant or plant parts is a vase with water comprising the tracer molecules.

30. Method as claimed in claim 17, wherein when said light is a laser visualization further comprises use of a filter to enhance specific fluorescence.

31. Method as claimed in claim 18, wherein said registration of the light is done by means of a camera.

32. Method as claimed in claim 1, wherein the tracer molecule has a CAS registration number selected from the group consisting of 2391-30-2, 477-73-6, 1562-90-9, 1829-00-1, 56509-06-9, 16470-24-9, 32694-95-4, 169762-28-1, 144470-48-4, 12270-53-0, 12270-53-0, 61968-72-7, 68444-86-0, 205265-33-4, 37299-86-8, 2321-07-5, 550-82-3, 2538-84-3, 65-61-2, 52237-03-3, 27344-41-8, and 6416-68-8.

33. Product as claimed in claim 23, wherein the tracer molecules are fluorescent tracer molecules.
